(19) United States
(12) Patent Application Publication
(10) Pub. No.: US 2026/0354875 A1
(43) Pub. Date: Nov. 12, 2026

(54) FIXATION PLATE FOR FRACTURE REPAIR

(71) Applicant: GLabs X, LLC, Redondo Beach, CA (US)

(72) Inventors: Ian Stine, Danville, CA (US); Anshu Singh, San Diego, CA (US); Mark Foley, Beverly, MA (US)

(21) Appl. No.: 19/130,521

(22) PCT Filed: Nov. 15, 2023
(86) PCT No.: PCT/US2023/079912

§ 371 Date: May 15, 2025

Related U.S. Application Data
(60) Provisional application No. 63/384,083, filed on Nov. 16, 2022.

(51) Int. Cl. A61B 17/80

(52) U.S. Cl. CPC A61B 17/8061; A61B 17/8085

(57) ABSTRACT

Bone surgical plate devices and methods for use in bone fracture surgeries, for example, clavicle fracture surgery. The systems and methods can include a plate device with specific design features including but not limited to differing designs, geometries, and configurations in multiple segments of the device.

### INCORPORATION BY REFERENCE TO ANY PRIORITY APPLICATIONS

This application claims priority to U.S. Provisional Patent Application No. 63/384,083, filed Nov. 16, 2022, which is incorporated herein by reference.

### BACKGROUND

### Field

Embodiments described herein relate to innovative surgical devices and methods that can be used to significantly improve clinical outcomes for patients while reducing healing times, reducing costs, and increasing surgical accuracy in bone fracture surgeries. Embodiments of bone surgical devices and methods can be particularly impactful on bone fracture surgeries, including, but not limited to, clavicle fracture surgery.

### SUMMARY

Certain embodiments described herein are directed to bone surgical systems, devices and methods of use, such as bone surgical fixation plates including but not limited to the use on the clavicle bone. Although certain embodiments may be described with respect to the clavicle bone, in other embodiments the same system, device or method having some or all of the features described for use with the clavicle bone may be used in systems, devices or methods for other bones. For purposes of this summary, certain aspects, advantages, and novel features of the invention are described herein. It is to be understood that not all such advantages necessarily may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

In some cases, a clavicle plate fixation system can include: a plate sized for positioning on a clavicle bone, the plate including: a central fastening segment; a medial connection segment coupled to a medial end of the central segment, the medial connection segment including a hole including two connected holes; a lateral connection segment coupled to a lateral end of the central fastening segment, the lateral connection segment including a hole including two connected holes; a medial fastening segment coupled to a medial end of the medial connection segment, the medial fastening segment including a first hole at a medial end of the medial fastening segment, a second hole at a lateral end of the medial fastening segment, and a third hole wherein the third hole is offset from the first hole and the second hole such that a portion of the third hole is past a first side of the medial fastening segment; a lateral fastening segment coupled to a lateral end of the lateral connection segment the lateral fastening segment including a fourth hole at a lateral end of the lateral fastening segment, a fifth hole at a medial end of the lateral fastening segment, and a sixth hole wherein the sixth hole is offset from the fourth hole and the fifth hole such that a portion of the sixth hole is past a second side of the medial fastening segment; wherein the medial fastening segment and the medial connection segment are separated by an indentation on a first side of the plate and an indentation on a second side of the plate, and the lateral fastening segment and the lateral connection segment are separated by a second indentation on the first side of the plate and a second indentation on the second side of the plate, and wherein the medial connection segment and the central fastening segment are separated by a third indentation on the first side of the plate and a third indentation on the second side of the plate, and the lateral connection segment and the central fastening segment are separated by a fourth indentation on the first side of the plate and a fourth indentation on the second side of the plate, wherein the third hole is offset from the first hole about a first axis by an angle of 45 degrees to 135 degrees, wherein the first axis extends from the lateral end of the medial fastening segment to the medial end of the medial fastening segment, and the sixth hole is offset from the fourth hole about a second axis by an angle of −45 degrees to −135 degrees, wherein the second axis extends from the medial end of the lateral fastening segment to the lateral end of the lateral fastening segment and wherein the plate is s-shaped along a longitudinal axis, wherein the longitudinal axis runs through the central fastening segment along a length of the plate.

In some cases, the plate is configured to be bent at a location of the indentations.

In some cases, the plate is configured to be coupled to a clavicle on a superior surface of the clavicle.

In some cases, the central fastening segment includes one or more screw holes.

In some cases each of the holes is a screw hole.

In some cases, the plate includes a thickness between 2 mm and 4 mm.

In some cases, a fixation plate system can include: a plate sized for positioning on a bone, the plate including: at least three fastening segments separated by an indentation on a first side of the plate and an indentation on a second side of the plate, each of the three fastening segments including one or more holes, wherein a medial segment of the at least three fastening segments includes a first tab offset from a top surface of the medial segment by an angle of 10 degrees to 135 degrees, and a lateral segment of the at least three fastening segments includes a second tab offset from a top surface of the medial segment by an angle of −10 degrees to −135 degrees.

In some cases, the first tab extends from a first side of the plate in a first direction, and the second tab extends from a second side of the plate in a second direction generally opposite the first direction.

In some cases, the medial segment does not comprise any tabs extending in the second direction from the second side of the plate.

In some cases, the lateral segment does not comprise any tabs extending in the first direction from the first side of the plate.

### BRIEF DESCRIPTION OF THE DRAWINGS

The drawings are provided to illustrate example embodiments and are not intended to limit the scope of the disclosure. A better understanding of the systems and methods described herein will be appreciated upon reference to the following description in conjunction with the accompanying drawings, wherein:

FIG. 1A illustrates a perspective view of a bone surgical plate device according to some embodiments herein.

FIG. 1B illustrates a front view of the bone surgical plate device according to some embodiments herein.

FIG. 1C illustrates a top view of the bone surgical plate device according to some embodiments herein.

FIG. 2A illustrates a top view of a bone surgical plate device according to some embodiments herein.

FIG. 2B illustrates a front view of a bone surgical plate device according to some embodiments herein.

FIG. 2C illustrates a perspective view of a bone surgical plate device according to some embodiments herein.

FIG. 2D illustrates a top view of a bone surgical plate device according to some embodiments herein.

FIG. 2E illustrates a front view of a bone surgical plate device according to some embodiments herein.

FIG. 2F illustrates a perspective view of a bone surgical plate device according to some embodiments herein.

FIG. 2G illustrates a top view of a bone surgical plate device according to some embodiments herein.

FIG. 2H illustrates a front view of a bone surgical plate device according to some embodiments herein.

FIG. 2I illustrates a back view of a bone surgical plate device according to some embodiments herein.

FIG. 2J illustrates a left side view of a bone surgical plate device according to some embodiments herein.

FIG. 2K illustrates a right side view of a bone surgical plate device according to some embodiments herein.

FIG. 2L illustrates a bottom view of a bone surgical plate device according to some embodiments herein.

FIG. 2M illustrates a perspective view of a bone surgical plate device according to some embodiments herein.

FIG. 3A illustrates a top view of a bone surgical plate device according to some embodiments herein.

FIG. 3B illustrates a front view of a bone surgical plate device according to some embodiments herein.

FIG. 3C illustrates a perspective view of a bone surgical plate device according to some embodiments herein.

FIG. 3D illustrates a top view of a bone surgical plate device according to some embodiments herein.

FIG. 3E illustrates a front view of a bone surgical plate device according to some embodiments herein.

FIG. 3F illustrates a perspective view of a bone surgical plate device according to some embodiments herein.

FIG. 3G illustrates a top view of a bone surgical plate device according to some embodiments herein.

FIG. 3H illustrates a front view of a bone surgical plate device according to some embodiments herein.

FIG. 3I illustrates a back view of a bone surgical plate device according to some embodiments herein.

FIG. 3J illustrates a left side view of a bone surgical plate device according to some embodiments herein.

FIG. 3K illustrates a right side view of a bone surgical plate device according to some embodiments herein.

FIG. 3L illustrates a bottom view of a bone surgical plate device according to some embodiments herein.

FIG. 3M illustrates a perspective view of a bone surgical plate device according to some embodiments herein.

FIG. 4A illustrates a top view of a bone surgical plate device according to some embodiments herein.

FIG. 4B illustrates a front view of a bone surgical plate device according to some embodiments herein.

FIG. 4C illustrates a perspective view of a bone surgical plate device according to some embodiments herein.

FIG. 4D illustrates a top view of a bone surgical plate device according to some embodiments herein.

FIG. 4E illustrates a front view of a bone surgical plate device according to some embodiments herein.

FIG. 4F illustrates a perspective view of a bone surgical plate device according to some embodiments herein.

FIG. 4G illustrates a top view of a bone surgical plate device according to some embodiments herein.

FIG. 4H illustrates a front view of a bone surgical plate device according to some embodiments herein.

FIG. 4I illustrates a back view of a bone surgical plate device according to some embodiments herein.

FIG. 4J illustrates a left side view of a bone surgical plate device according to some embodiments herein.

FIG. 4K illustrates a right side view of a bone surgical plate device according to some embodiments herein.

FIG. 4L illustrates a bottom view of a bone surgical plate device according to some embodiments herein.

FIG. 4M illustrates a perspective view of a bone surgical plate device according to some embodiments herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto or as pursued in the future is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present technology. The section headings used herein are merely provided to enhance readability and are not intended to limit the scope of the embodiments disclosed in a particular section to the features or elements disclosed in that section.

### Bone Surgical Plate Devices

Bone surgical plate devices as described herein, such as for use on the clavicle bone or for other bones, may include one or more of the following features. In some embodiments, a bone surgical plate device can include a plurality of screw holes. In some embodiments, at least one offset screw can be offset from an exterior surface, a main plane, and/or a longitudinal axis extending from a medial end of the bone surgical plate to a lateral end of the bone surgical plate such that the at least one offset screw provides multiplanar bone fixation. In some embodiments, the bone surgical plate device can include a higher density of screw holes (i.e., a higher number of screw holes across a length of the bone surgical plate). In some embodiments, the higher density of screw holes, a position of the screw holes, and the at least one offset screw can provide a practitioner a selection of entry angles based on the anatomy of a patient. For example, in clavicle fixation procedures, the patient's head, nerves, fracture pattern, and blood vessels can make certain angles of entry problematic or difficult. In some embodiments, the higher density of the screw holes, the position of the screw holes, and the selection of entry angles can make fixation of the bone surgical plate device by a practitioner faster, safer, and more effective.

In some embodiments, the higher density of the screw holes, the position of the screw holes, and the at least one offset screw can allow a practitioner more optimally utilize a density of a bone and/or dimensions of the bone. In some embodiments, the at least one offset screw can allow the bone surgical plate device to be shorter and/or thinner than bone surgical plate devices without the at least one offset screw while providing a same fixation strength or a greater fixation strength.

In some embodiments, a shorter length of the bone surgical plate device can reduce a size of an incision required for fixation of the bone surgical plate device, simplify placement of the bone surgical plate device, and/or provide improved bone contouring.

In some embodiments, a thickness or height of the bone surgical plate device can vary along the length of the bone surgical plate device. In some embodiments, a smaller thickness of the bone surgical plate device and/or the varying thickness can provide more patient comfort, lower removal rates, and easier plate contouring for the practitioner during fixation or implantation of the bone surgical plate device.

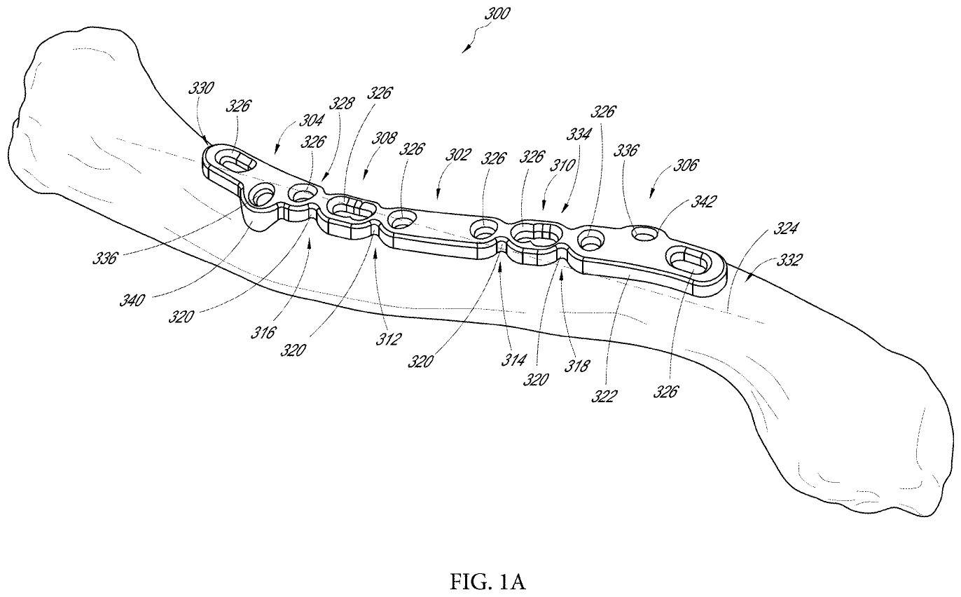

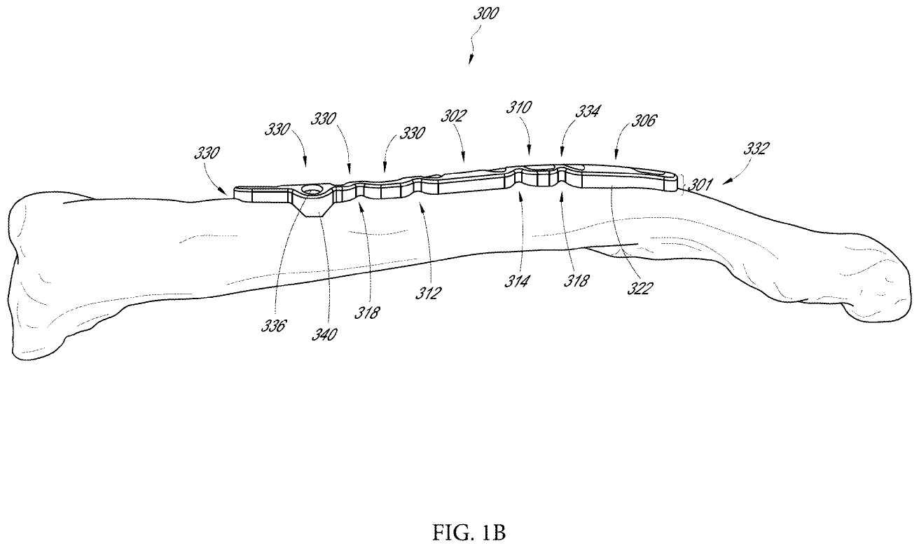

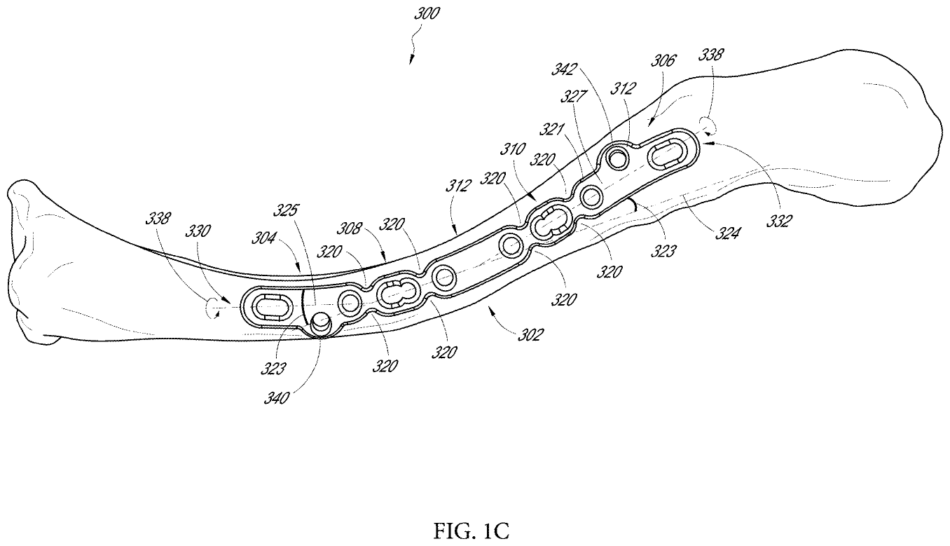

Further details of bone surgical plate devices incorporating one or more of the features described above will be further described with respect to tabular segment plates as shown in FIGS. 1A-4M.

Further details are of bone surgical plate devices described herein are found in U.S. Pat. No. 11,337,739B2, which is incorporated herein by reference.

### Tabular Segment Plate

FIGS. 1A-1C illustrate an embodiment of a bone surgical plate device 300. The bone surgical plate device 300 can include a central fastening segment 302, a medial fastening segment 304, a lateral fastening segment 306, a medial connection segment 308, and a lateral connection segment 310. In some cases, the bone surgical plate device 300 can include a thickness 301. The thickness 301 can be between about 1 mm and 4 mm. The thickness 301 can allow for the bone surgical plate device 300 to be bent to a shape of a bone. In some cases, the thickness 301 can vary across a length of bone surgical plate device 300. In some cases, the thickness 301 can vary across one or more of the central fastening segment 302, the medial fastening segment 304, the lateral fastening segment 306, the medial connection segment 308, and the lateral connection segment 310 can each comprise a same thickness 301. In some cases, the central fastening segment 302, the medial fastening segment 304, the lateral fastening segment 306, the medial connection segment 308, and/or the lateral connection segment 310 can each comprise a same thickness 301. In some cases, the central fastening segment 302, the medial fastening segment 304, the lateral fastening segment 306, the medial connection segment 308, and/or the lateral connection segment 310 can each comprise a different thickness 301. In some embodiments, a thickness 301 of the central fastening segment 302 can be greater than a thickness 301 of the medial fastening segment 304 and/or the lateral fastening segment 306.

In some cases, the medial connection segment 308 can be coupled to a medial end 312 of the central fastening segment 302, and the lateral connection segment 310 can be coupled to a lateral end 314 of the central fastening segment 302. In some cases, the medial fastening segment 304 can be coupled to a medial end 316 of the medial connection segment 308, and the lateral fastening segment 306 can be coupled to a lateral end 318 of the lateral connection segment 310.

In some cases, the central fastening segment 302, the medial fastening segment 304, the lateral fastening segment 306, the medial connection segment 308, and the lateral connection segment 310 can be removably coupled to each other such that various shapes and sizes of the central fastening segment 302, the medial fastening segment 304, the lateral fastening segment 306, the medial connection segment 308, and the lateral connection segment 310 can be coupled together to change the size and shape of the bone surgical plate device 300 to fit a clavicle bone. In other cases, the central fastening segment 302, the medial fastening segment 304, the lateral fastening segment 306, the medial connection segment 308, and the lateral connection segment 310 can be permanently coupled to each other such that the central fastening segment 302, the medial fastening segment 304, the lateral fastening segment 306, the medial connection segment 308, and the lateral connection segment 310 form a single piece of material.

In some cases, as best shown in FIG. 1C, the bone surgical plate device 300 can include one or more indentations 320. The bone surgical plate device 300 can include one or more indentations 320 on a first side 321 and one or more indentations 320 on a second side 322. In some cases, the one or more indentations 320 can separate the central fastening segment 302 and the medial connection segment 308, the central fastening segment 302 and the lateral connection segment 310, the medial connection segment 308 and the medial fastening segment 304, and the lateral connection segment 310 and the lateral fastening segment 306. In some cases, the bone surgical plate device 300 can be bent at a location of each of the one or more indentations 320. In this way, the bone surgical plate device 300 can be bent to be a same or similar shape of a surface of the clavicle bone and/or any other bone. In some cases, the indentations 320 can allow the bone surgical plate device 300 to be bent when patient anatomy is unique, and/or a fracture pattern call for usage of an off-side plate for the plate for the patient's height.

In some cases, the bone surgical plate device 300 can include a curved configuration. The medial connection segment 308 can extend from the medial end 312 of the central fastening segment 302 in a medial direction such that a portion of the first side 321 of the bone surgical plate device 300 along the medial connection segment 308 is at an angle 323 from a longitudinal axis 324. The longitudinal axis 324 can run through the central fastening segment 302 along a length of the bone surgical plate device 300. In some cases, the medial fastening segment 304 can extend from the medial end 316 of the medial connection segment 308 in the medial direction such that a portion of the first side 321 of the bone surgical plate device 300 along the medial fastening segment 304 is at the angle 323 from the longitudinal axis 324. In some cases, the lateral connection segment 310 can extend from the lateral end 314 of the central fastening segment 302 in a lateral direction such that a portion of the second side 322 of the bone surgical plate device 300 along the lateral connection segment 310 is at the angle 323 from the longitudinal axis 324. In some cases, the lateral fastening segment 306 can extend from the lateral end 318 if the lateral connection segment 310 in the lateral direction such that a portion of the second side 322 of the bone surgical plate device 300 along the lateral fastening segment 306 is at the angle 323 from the longitudinal axis 324. The angle 323 can be between 0 degrees and 90 degrees. In some cases, the angle 323 can be about 45 degrees.

In some cases, as best shown in FIG. 1C the medial fastening segment 304, the lateral fastening segment 306, the medial connection segment 308, and/or the lateral connection segment 310 can include a curved configuration such that the medial fastening segment 304, the lateral fastening segment 306, the medial connection segment 308, and/or the lateral connection segment 310 are curved towards or away from the longitudinal axis 324. In some cases, the bone surgical plate device 300 can be s-shaped along the longitudinal axis 324 so the bone surgical plate device 300 can be the same or similar shape of the superior surface of the clavicle bone and/or any other bone. In some cases, the bone surgical plate device 300 can be the same or similar shape of any surface of the clavicle bone.

In some cases, as best shown in FIG. 1C, the central fastening segment 302 can include one or more holes 326. In some cases, the central fastening segment 302 can include between 1 and 6 holes 326. In some cases, one or more of the one or more holes 326 can be a screw hole. In these cases, one or more of the one or more holes 326 can be hole windows and can be oval shaped such that a distance across each of the one or more of the one or more holes 326 along a length of the bone surgical plate device 300 can be longer than a distance across the one or more of the one or more holes 326 perpendicular to the distance across the one or more of the one or more holes 326 along the length of the bone surgical plate device 302. In some cases, the oval shape of the one or more of the one or more holes 326 or hole windows can provide a practitioner with a plurality of location and angles of entry when inserting a screw into a bone through the one or more of the one or more screw holes 326. In other cases, the one or more holes 326 can reduce a weight of the central fastening segment 302. In some cases, one or more of the one or more holes 326 can be recessed from a top surface of the central fastening segment 302 such that in the cases where one or more of the one or more holes 326 are a screw hole, when a screw is inserted into the one or more holes 326 a top surface of the screw is flush with the top surface of the central fastening segment 302. In some cases, the central fastening segment 302 can include two holes 326 and the two holes 326 can be located at the medial end 312 and the lateral end 314 of the central fastening segment 302.

In some cases, the medial connection segment 308 and the lateral connection segment 310 can each include one or more holes 326. In some cases, the medial connection segment 308 and the lateral connection segment 310 can include between 1 and 6 holes 326. In some cases, one or more of the one or more holes 326 can be a screw hole. In other cases, the one or more holes 326 can reduce a weight of the medial connection segment 308 and a weight of the lateral connection segment 310. In some cases, one or more of the one or more holes 326 can be recessed from a top surface of the medial connection segment 308 and a top surface of the lateral connection segment 310 such that in the cases where one or more of the one or more holes 326 are a screw hole, when a screw is inserted into the one or more holes 326 a top surface of the screw is flush with the top surface of the medial connection segment 308 and the top surface of the lateral connection segment 310. In these cases, as best shown in FIG. 1C, one or more of the one or more holes 326 can be hole windows and can be oval shaped such that a distance across each of the one or more of the one or more holes 326 along an axis 320 (as defined below) or an axis 327 (as defined below) can be longer than a distance across the one or more of the one or more holes 326 perpendicular to the axis 325 or the axis 327. In some cases, the oval shape of the one or more of the one or more holes 326 or hole windows can provide a practitioner with a plurality of location and angles of entry when inserting a screw into a bone through the one or more of the one or more screw holes 326. In some cases, the medial connection segment 308 and the lateral connection segment 310 can each include two holes 326 and the two holes 326 can be connected to form one oval shaped hole.

In some cases, the medial fastening segment 304 and the lateral fastening segment 306 can each include one or more holes 326. In some cases, the medial fastening segment 304 and the lateral fastening segment 306 can include between 1 and 6 holes 326. In some cases, one or more of the one or more holes 326 can be a screw hole. In these cases, as best shown in FIG. 1C, one or more of the one or more screw holes 326 can be screw hole windows and can be oval shaped such that a distance across each of the one or more of the one or more screw holes 326 along an axis 325 (as defined below) or an axis 327 (as defined below) can be longer than a distance across the one or more of the one or more screw holes 326 perpendicular to the axis 325 or the axis 327. The oval shape of the one or more of the one or more screw holes 326 or screw hole windows can provide a practitioner with a plurality of location and angles of entry when inserting a screw into a bone through the one or more of the one or more screw holes 326. In other cases, the one or more holes 326 can reduce a weight of the medial fastening segment 304 and a weight of the lateral fastening segment 306. In some cases, one or more of the one or more holes 326 can be recessed from a top surface of the medial fastening segment 304 and a top surface of the lateral fastening segment 306 such that in the cases where one or more of the one or more holes 326 are a screw hole, when a screw is inserted into the one or more holes 326 a top surface of the screw is flush with the top surface of the medial fastening segment 304 and the top surface of the lateral fastening segment 306.

In some cases, the medial fastening segment 304 can include two holes 326. A first hole 326 can be located at a lateral end 328 of the medial fastening segment 304 and a second hole 326 can be located at a medial end 330 of the medial fastening segment 304. In some cases, the first hole 326 can be oval shaped.

In some cases, the lateral fastening segment 306 can include two holes 326. A first hole 326 can be located at a lateral end 332 of the lateral fastening segment 306 and a second hole 326 can be located at a medial end 334 of the lateral fastening segment 306. In some cases, the second hole 326 can be oval shaped.

In some cases, the medial fastening segment 304 and the lateral fastening segment 306 can each include a tab with an offset hole 336, wherein the tab extends past a side of the bone surgical plate 300. In some cases, the tab referred to herein may be a tabular plane. The tab and/or the offset hole 336 of the medial fastening segment 304 can be offset from an axis 325 at an angle 338 about the axis 325. The axis 325 can extend from the medial end 316 to the medial end 330. The tab and/or the offset hole 336 of the medial fastening segment 304 can be offset by the angle 338 of 10 degrees to 45 degrees or 45 degrees to 135 degrees. The tab and/or the offset hole 336 of the lateral fastening segment can be offset from an axis 327 at an angle 338 about the axis 327. The axis 325 can extend from the lateral end 318 to the lateral end 332. The tab and/or the offset hole 336 of the lateral fastening segment 306 can be offset by the angle 338 of −45 degrees to −135 degrees.

In some cases, at least a portion of the offset hole 336 of the medial fastening segment 304 can be anterior to the first side 321 of the bone surgical plate device 300 and the medial fastening segment 304 can include a curved portion 340. The curved portion 340 can form an anterior side of the offset hole 336 and can form a continuous surface with the first side 321 of the bone surgical plate device 300.

In some cases, at least a portion of the offset hole 336 of the lateral fastening segment 306 can be posterior to the second side 322 of the bone surgical plate device 300 and the lateral fastening segment 306 can include a curved portion 342. The curved portion 342 can form a posterior side of the offset hole 336 and can form a continuous surface with the second side 322 of the bone surgical plate device 300.

In some cases, one or more holes 326 and the offset hole 336 can be threaded screw holes to allow screws to fasten the bone surgical plate device 300 to the bone. In some cases, non-threaded screw holes can be used. In some cases, one or more holes 326 can be cortex screw holes. In some cases, one or more holes 326 can be k-wire holes. In some cases, the offset configuration of the offset hole 336 can allow for a strategic screw placement that can use smaller screws, thinner screws, and/or less screws to fasten the bone surgical plate device 300 to the bone. Smaller screws and less screws can be used due to the increased stability provided by the multiplanar configuration.

In some cases, a portion of the bone surgical plate device 300 around one or more of the holes 326 and/or the offset holes 336 can include a thickness 301 that is greater than a thickness 301 of the rest of the bone surgical plate device 300 such that a top surface of the bone surgical plate device 300 at the portion of the bone surgical plate device 300 around one or more of the holes and/or the offset holes 336 is raised or mound shaped. The raised or mound shaped portion around one or more of the holes 326 and/or the offset holes 336 can increase a strength of the bone surgical plate device 300 at or about the one or more of the holes 326 and/or the offset holes 336 decreasing failure of the bone surgical plate device 300 at or about the one or more of the holes 326 and/or the offset holes 336.

All existing, well-known, plate or implant fastening devices and/or techniques can be used to fasten the bone surgical plate device 300 to the bone.

The bone surgical plate device 300 can be used in various bone procedures. For example, the bone surgical plate device 300 can be used in a clavicle fracture procedure.

Although some details, geometries, and configurations of the bone surgical plate device 300 described herein with respect to clavicle fracture surgery, the bone surgical plate device 300 can also be used in other types of bone surgeries. For example, the bone surgical plate device 300 described herein can be used for ulna surgery.

The bone surgical plate device 300 can provide an increased stability which can lessen the need for removal or second surgery. Additionally, the bone surgical plate device 300 can be shorter in length than traditional implants which can allow for a smaller incision and less scaring. Additionally, the bone surgical plate device 300 can cause less patient discomfort and faster/better healing. The bone surgical plate device 300 can be less expensive and can reduce the need for multiple surgeries. In some embodiments, the bone surgical plate 300 can simplify the surgical procedure reducing the likelihood of mistakes. The bending feature of the bone surgical plate device 300 can assist the device in adjusting to the anatomy and provide a more tailored and personalized implant device.

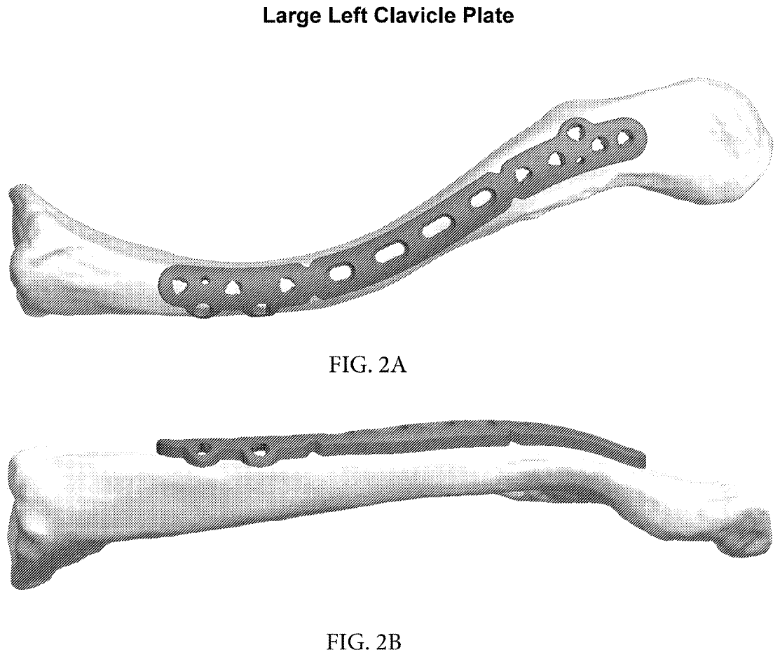

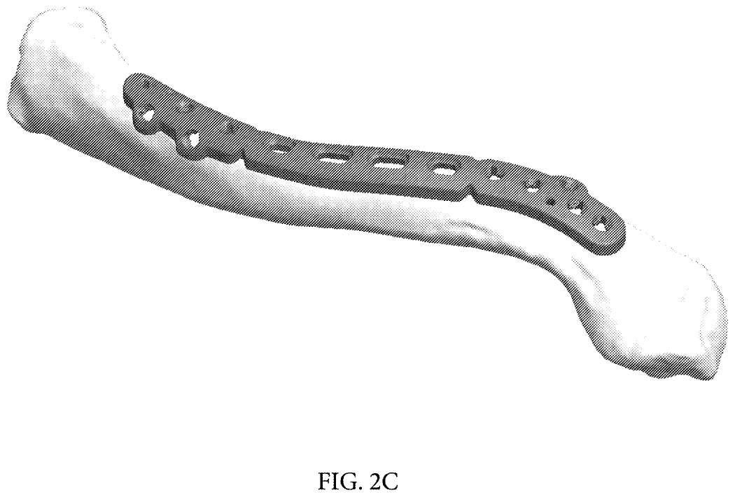

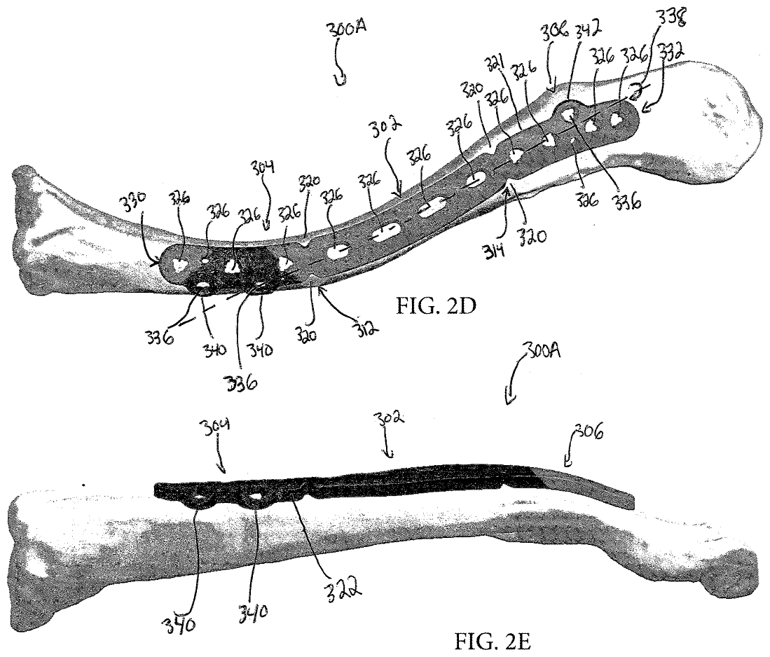

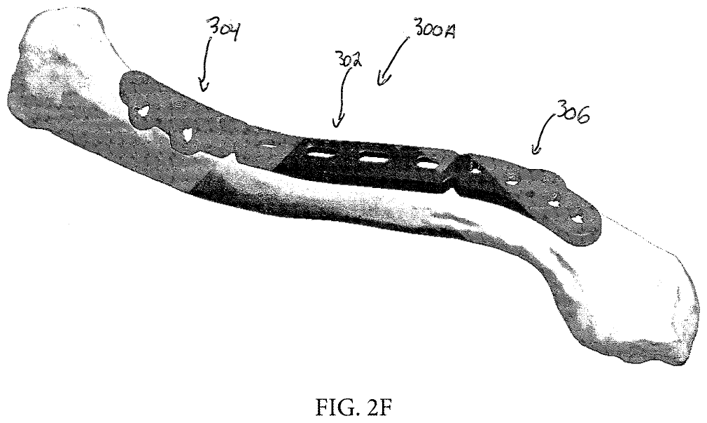

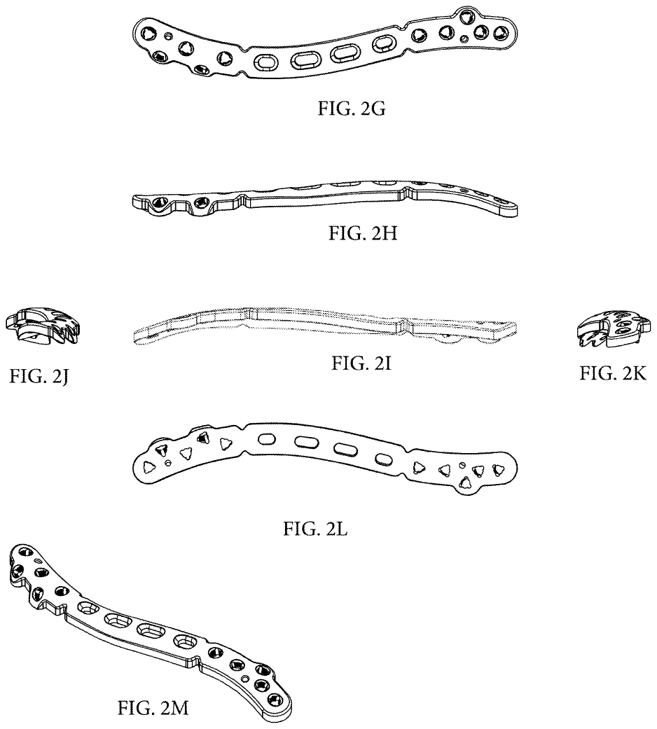

FIGS. 2A-2F, 3A-3F, and 4A-4F illustrate further embodiments of the bone surgical plate device 300A, 300B and 300C. FIGS. 2G-2M, 3G-3M, and 4G-4M show alternative views of the bone surgical plate devices 300A, 300B, and 300C. In some cases, the bone surgical plate devices 300A, 300B, and 300C can be used for different sizes of clavicle bones (e.g., large, medium and small clavicle bones). In some cases, the bone surgical plate device 300A can be used for large clavicle bones. In some cases, the bone surgical plate device 300B can be used for medium clavicle bones. In some cases, the bone surgical plate device 300C can be used for small clavicle bones.

The bone surgical plate devices 300A, 300B, and 300C can include a central fastening segment 302, a medial fastening segment 304, and a lateral fastening segment 306. In some cases, the bone surgical plate devices 300A, 300B, and 300C can include a thickness 301. The thickness 301 can be between about 1 mm and 4 mm. The thickness 301 can allow for the bone surgical plate devices 300A, 300B, and 300C to be bent to a shape of a bone. In some cases, the thickness 301 can vary across a length of bone surgical plate devices 300A, 300B, and 300C. In some cases, the thickness 301 can vary across one or more of the central fastening segment 302, the medial fastening segment 304, and/or the lateral fastening segment 306 can each comprise a same thickness 301. In some cases, the central fastening segment 302, the medial fastening segment 304, and/or the lateral fastening segment 306 can each comprise a same thickness 301. In some cases, the central fastening segment 302, the medial fastening segment 304, and/or the lateral fastening segment 306 can each comprise a different thickness 301. In some embodiments, a thickness 301 of the central fastening segment 302 can be greater than a thickness 301 of the medial fastening segment 304 and/or the lateral fastening segment 306.

In some cases, the medial fastening segment 304 can be coupled to a medial end 312 of the central fastening segment 302, and the lateral fastening segment 306 can be coupled to a lateral end 314 of the central fastening segment 302.

In some cases, the central fastening segment 302, and the medial fastening segment 304, the lateral fastening segment 306, can be removably coupled to each other such that various shapes and sizes of the central fastening segment 302, the medial fastening segment 304, and the lateral fastening segment 306 can be coupled together to change the size and shape of the bone surgical plate devices 300A, 300B, and 300C to fit a clavicle bone and/or any other bone. In other cases, the central fastening segment 302, the medial fastening segment 304, and the lateral fastening segment 306 can be permanently coupled to each other such that the central fastening segment 302, the medial fastening segment 304, and the lateral fastening segment 306 form a single piece of material.

In some cases, as best shown in FIGS. 2D, 3D, and 4D, the bone surgical plate devices 300A, 300B, and 300C can include one or more indentations 320. The bone surgical plate devices 300A, 300B, and 300C can include one or more indentations 320 on a first side 321 and one or more indentations 320 on a second side 322. In some cases, the one or more indentations 320 can separate the central fastening segment 302 and the medial connection segment 308, the central fastening segment 302 and the lateral connection segment 310, the medial connection segment 308 and the medial fastening segment 304, and the lateral connection segment 310 and the lateral fastening segment 306. In some cases, the bone surgical plate devices 300A, 300B, and 300C can be bent at a location of each of the one or more indentations 320. In this way, the bone surgical plate devices 300A, 300B, and 300C can be bent to be a same or similar shape of a surface of the clavicle bone and/or any other bone. In some cases, the indentations 320 can allow the bone surgical plate devices 300A, 300B, and 300C to be bent when patient anatomy is unique, and/or a fracture pattern call for usage of an off-side plate for the plate for the patient's height.

In some cases, as best shown in FIGS. 2D, 3D, and 4D, the central fastening segment 302, the medial fastening segment 304, and/or the lateral fastening segment 306 can include a curved configuration such that the central fastening segment 302, the medial fastening segment 304, and/or the lateral fastening segment 306, are curved towards or away from a longitudinal axis 324. The longitudinal axis 324 can run through the central fastening segment 302 along a length of the bone surgical plate device 300. In some cases, the bone surgical plate devices 300A, 300B, and 300C can be s-shaped along the longitudinal axis 324 so the bone surgical plate devices 300A, 300B, and 300C can be the same or similar shape of the surface of the clavicle bone and/or any other bone. In some cases, the bone surgical plate device 300 can be the same or similar shape of any surface of the clavicle bone.

In some cases, as best shown in FIGS. 2A, 3A, and 4A, the central fastening segment 302 can include one or more holes 326. In some cases, the central fastening segment 302 can include between 1 and 6 holes 326. In some cases, one or more of the one or more holes 326 can be a screw hole. In these cases, one or more of the one or more holes 326 can be hole windows and can be oval shaped such that a distance across each of the one or more of the one or more holes 326 along a length of the bone surgical plate devices 300A, 300B, and 300C can be longer than a distance across the one or more of the one or more holes 326 perpendicular to the distance across the one or more of the one or more holes 326 along the length of the bone surgical plate device 302. In some cases, the oval shape of the one or more of the one or more holes 326 or hole windows can provide a practitioner with a plurality of location and angles of entry when inserting a screw into a bone through the one or more of the one or more screw holes 326. In other cases, the one or more holes 326 can reduce a weight of the central fastening segment 302. In some cases, one or more of the one or more holes 326 can be recessed from a top surface of the central fastening segment 302 such that in the cases where one or more of the one or more holes 326 are a screw hole, when a screw is inserted into the one or more holes 326 a top surface of the screw is flush with the top surface of the central fastening segment 302. In some cases, the central fastening segment 302 can include four holes 326.

In some cases, the medial fastening segment 304 and the lateral fastening segment 306 can each include one or more holes 326. In some cases, the medial fastening segment 304 and the lateral fastening segment 306 can include between 1 and 6 holes 326. In some cases, one or more of the one or more holes 326 can be a screw hole. In these cases, one or more of the one or more holes 326 can be hole windows and can be oval shaped such that a distance across each of the one or more of the one or more holes 326 along a length of the bone surgical plate devices 300A, 300B, and 300C can be longer than a distance across the one or more of the one or more holes 326 perpendicular to the distance across the one or more of the one or more holes 326 along the length of the bone surgical plate device 302. The oval shape of the one or more of the one or more screw holes 326 or screw hole windows can provide a practitioner with a plurality of location and angles of entry when inserting a screw into a bone through the one or more of the one or more screw holes 326. In other cases, the one or more holes 326 can reduce a weight of the medial fastening segment 304 and a weight of the lateral fastening segment 306. In some cases, one or more of the one or more holes 326 can be recessed from a top surface of the medial fastening segment 304 and a top surface of the lateral fastening segment 306 such that in the cases where one or more of the one or more holes 326 are a screw hole, when a screw is inserted into the one or more holes 326 a top surface of the screw is flush with the top surface of the medial fastening segment 304 and the top surface of the lateral fastening segment 306.

In some cases, as shown in FIGS. 2A-2M, the medial fastening segment 304 can include four holes 326. In some cases, at least one of the four holes 326 can be smaller than the remaining four holes 326.

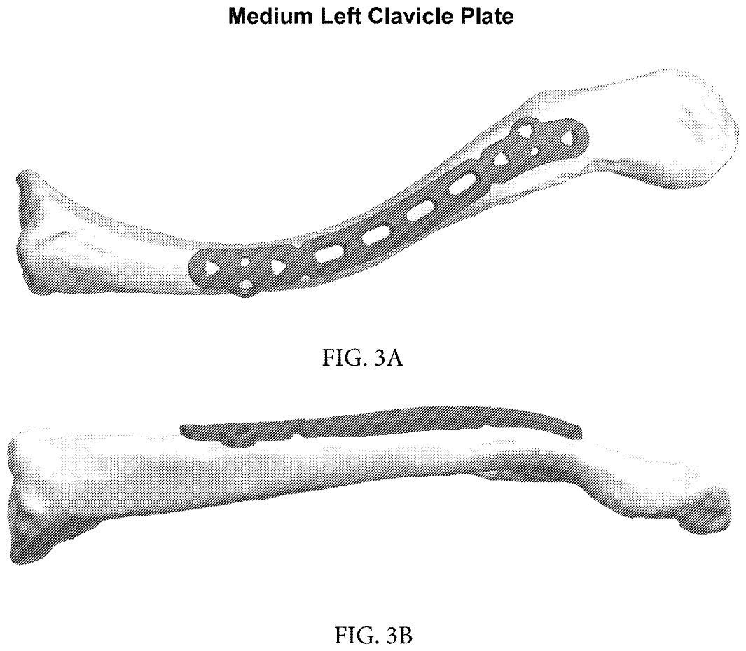

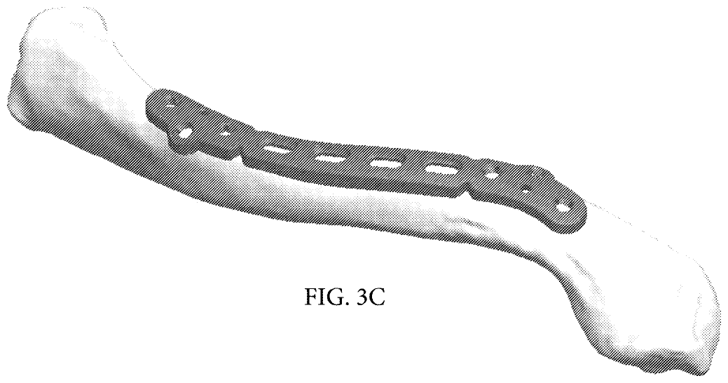

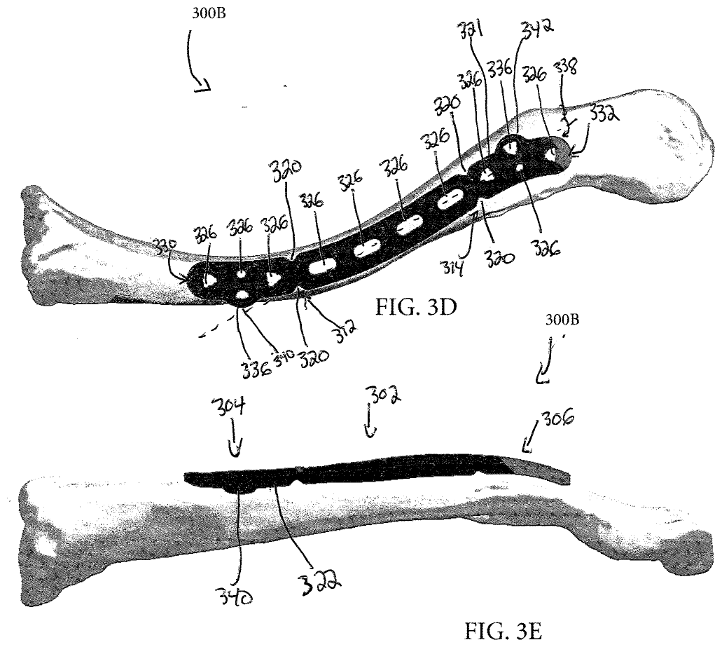

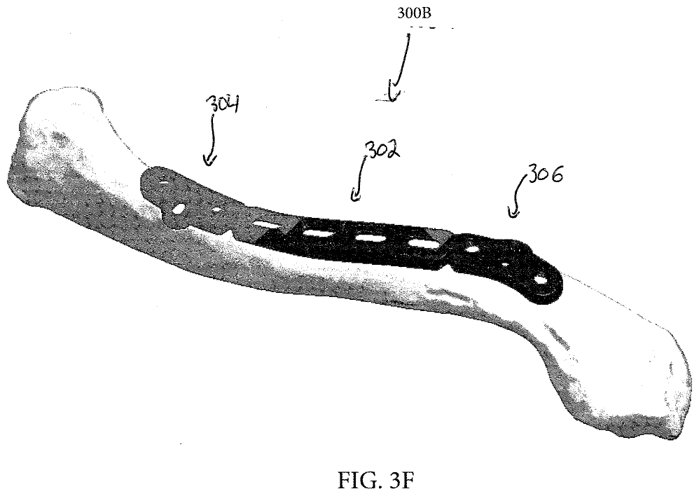

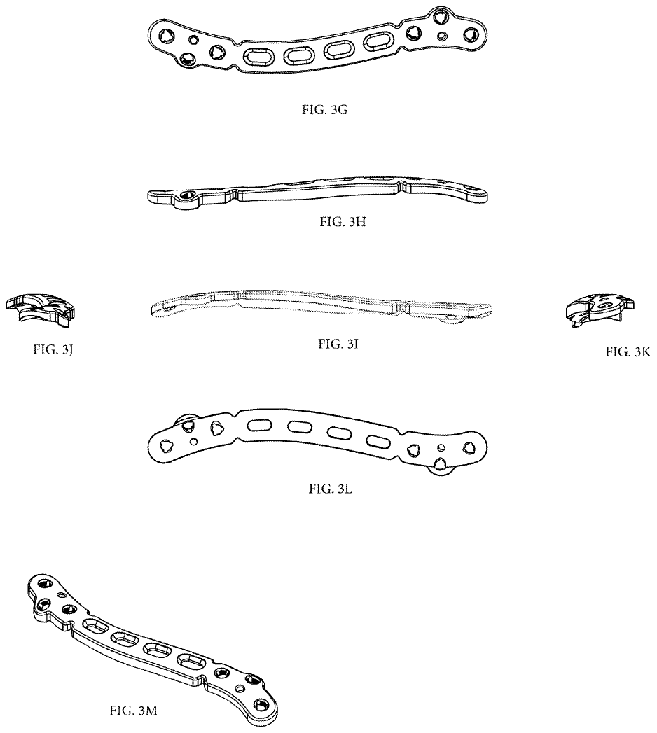

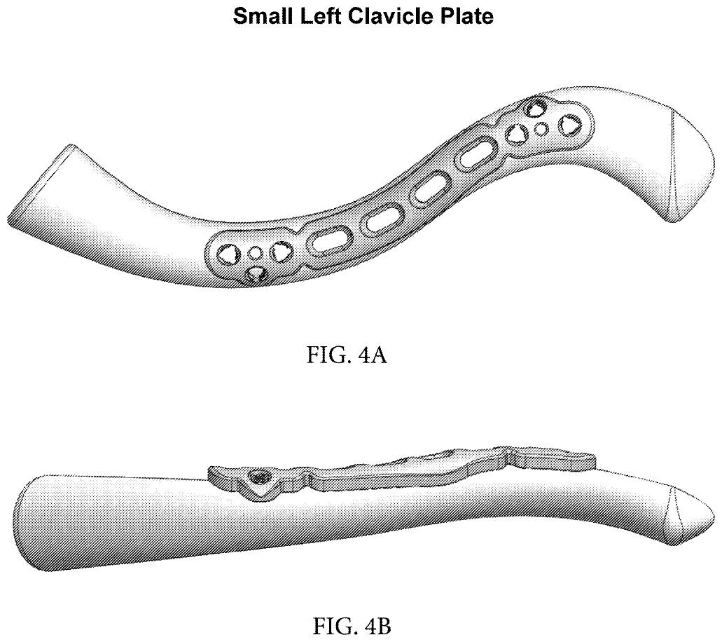

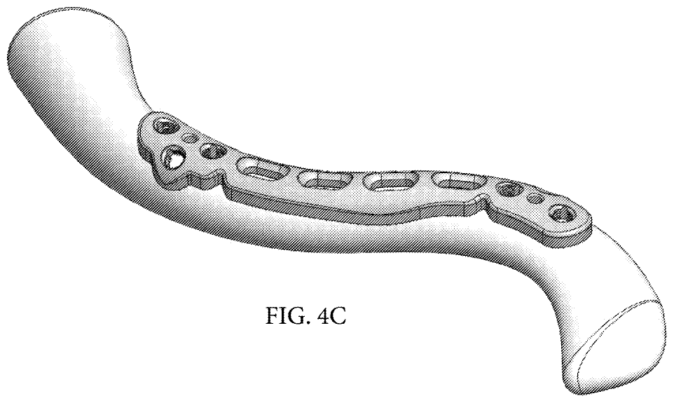

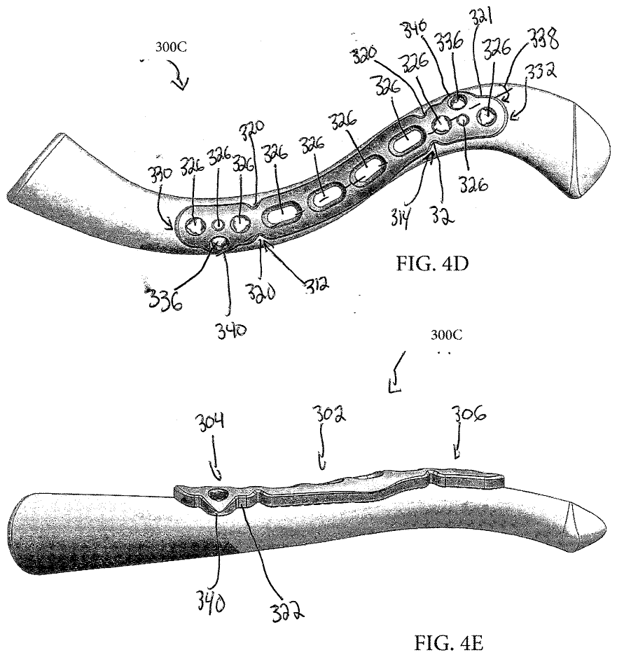

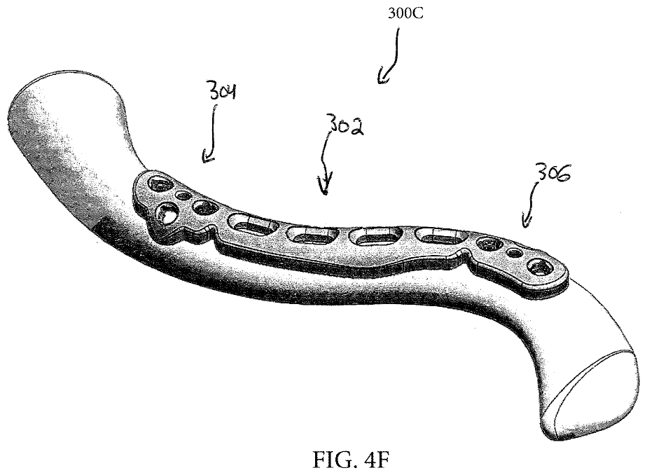

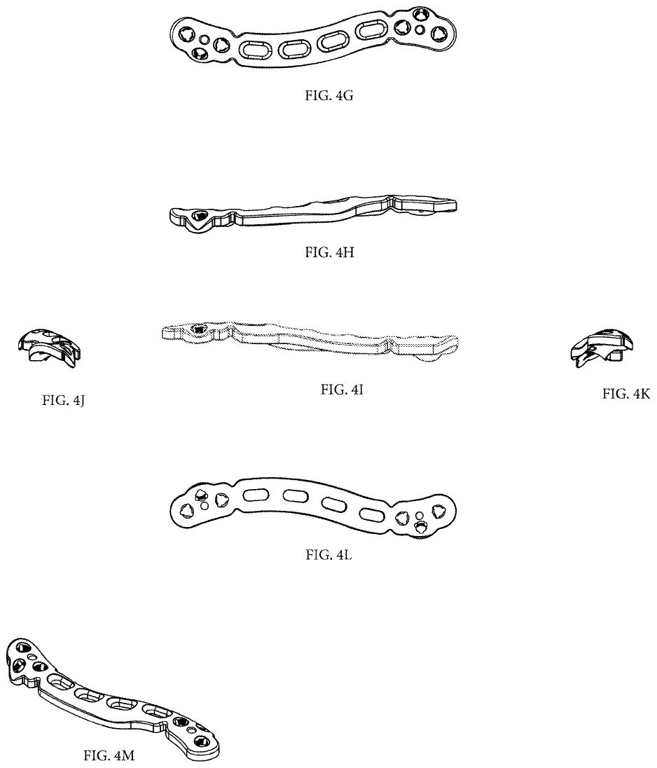

In some cases, as shown in FIGS. 3A-4M, the medial fastening segment 304 can include three holes 326. In some cases, at least one of the three holes 326 can be smaller than the remaining three holes 326.

In some cases, as shown in FIGS. 2A-2M, the lateral fastening segment 306 can include five holes 326. In some cases, at least one of the five holes 326 can be smaller than the remaining five holes 326.

In some cases, as shown in FIGS. 3A-4M, the lateral fastening segment 306 can include three holes 326. In some cases, at least one of the three holes 326 can be smaller than the remaining five holes 326.

In some cases, a number of holes 326 of the central fastening segment 302, the medial fastening segment 304, and/or the lateral fastening segment 306 can depend on a length of the bone surgical plate devices 300A, 300B, and 300C. In some cases, a bone surgical plate device 300A with a longer length, as shown in FIGS. 2A-2M can include more holes 326 than bone surgical plate devices 300B and 300C with a shorter length, as shown in FIGS. 3A-4M. In some cases, a location and/or spacing of the holes 326 can depend on the length of the bone surgical plate devices 300A, 300B, and 300C.

In some cases, the bone surgical plate device 300A can include a length of about 100-130 mm. In some cases, the bone surgical plate device 300A can include a length of about 115 mm. In some cases, the bone surgical plate device 300B can include a length of about 80-115 mm. In some cases, the bone surgical plate device 300B can include a length of about 100 mm. In some cases, the bone surgical plate device 300C can include a length of about 60-100 mm. In some cases, the bone surgical plate device 300C can include a length of about 80 mm.

In some cases, the medial fastening segment 304 and the lateral fastening segment 306 can each include a tab with the offset hole 336, wherein the tab extends past sides of the bone surgical plate devices 300A, 300B, and 300C. In some cases, the tab referred to herein may be a tabular plane. The tab and/or the offset hole 336 of the medial fastening segment 304 and/or the lateral fastening segment 306 can be offset from a longitudinal axis 324 at an angle 338 about the longitudinal axis 324. The tab and/or the offset hole 336 of the medial fastening segment 304 can be offset by the angle 338 of 10 degrees to 45 degrees or 45 degrees to 135 degrees. The tab and/or the offset hole 336 of the lateral fastening segment 306 can be offset by the angle 338 of −10 degrees to −45 degrees or −45 degrees to 135 degrees.

In some cases, the tab of the medial fastening segment 306 can extend from the first side 321 of the bone surgical plate devices 300A, 300B, and 300C. In some cases, at least a portion of the tab and/or offset hole 336 of the medial fastening segment 304 can be anterior to the first side 321 of the bone surgical plate devices 300A, 300B, and 300C and the tab can include a curved portion 340. The curved portion 340 can form an anterior side of the tab and/or offset hole 336 and can form a continuous surface with the first side 321 of the bone surgical plate devices 300A, 300B, and 300C.

In some cases, the tab of the lateral fastening segment 306 can extend from the second side 322 of the bone surgical plate devices 300A, 300B, and 300C. In some cases, at least a portion of the tab and/or the offset hole 336 of the lateral fastening segment 306 can be posterior to the second side 322 of the bone surgical plate devices 300A, 300B, and 300C and the tab can include a curved portion 342. The curved portion 342 can form a posterior side of the tab and/or offset hole 336 and can form a continuous surface with the second side 322 of the bone surgical plate devices 300A, 300B, and 300C.

In some cases, the medial fastening segment 304 and the lateral fastening segment 306 can each include a plurality of tabs with offset holes 336 or a plurality of tabular planes with offset holes 336. In some cases, all of the plurality of tabs with offset holes 336 of the medial fastening segment 304 can extend from the first side 321 of the bone surgical plate devices 300A, 300B, and 300C, such that at least a portion each of the plurality of tabs and/or offset holes 336 can be anterior to the first side 321 of the bone surgical plate device 300A, 300B, and 300C. In some cases, the medial fastening segment 304 does not include any tabs and/or offset holes 336 that extend from the second side 322 of the bone surgical plate devices 300A, 300B, and 300C, or the medial fastening segment 304 does not include any tabs and/or offset holes 336 that extend posterior to the second side 322 of the bone surgical plate devices 300A, 300B, and 300C. In some cases, all of the plurality of tabs with offset holes 336 of the lateral fastening segment 306 can extend from the second side 321 of the bone surgical plate devices 300A, 300A, and 300C, such that at least a portion of the plurality of tabs and/or the offset holes 336 of the lateral fastening segment 306 can be posterior to the second side 322 of the bone surgical plate devices 300A, 300B, and 300C. In some cases, the lateral fastening segment 306 does not include any tabs and/or offset holes 336 that extend from the first side 321 of the bone surgical plate devices 300A, 300B, and 300C, or the lateral fastening segment 306 does not include any tabs and/or offset holes 336 that extend anterior to the first side 321 of the bone surgical plate devices 300A, 300B, and 300C. In some cases, a number of offset holes 336 of the medial fastening segment 304 and the lateral fastening segment 306 can be based on or depend on the length of the bone surgical plate devices 300A, 300B, and 300C. In some cases, as best shown in FIGS. 3D-3F, the medical fastening segment 304 can include two offset holes or tabular planes with offset holes 336.

In some cases, one or more holes 326 and the offset hole 336 can be threaded screw holes to allow screws to fasten the bone surgical plate device 300 to the bone. In some cases, non-threaded screw holes can be used. In some cases, one or more holes 326 can be cortex screw holes. In some cases, one or more holes 326 can be k-wire holes. In some cases, the offset configuration of the offset hole 336 can allow for a strategic screw placement that can use smaller screws, thinner screws, and/or less screws to fasten the bone surgical plate devices 300A, 300B, and 300C to the bone. Smaller screws and less screws can be used due to the increased stability provided by the multiplanar configuration. In some cases, one or more holes 326 can form a 90-degree angle with the top surface of the bone surgical plate devices 300 and 300A. In some cases, one or more holes 326 can form an angle between 0 degrees and 90 degrees with the top surface of the bone surgical plate devices 300 and 300A.

In some cases, a portion of the bone surgical plate device 300 around one or more of the holes 326 and/or the offset holes 336 can include a thickness 301 that is greater than a thickness 301 of the rest of the bone surgical plate device 300 such that a top surface of the bone surgical plate device 300 at the portion of the bone surgical plate device 300 around one or more of the holes and/or the offset holes 336 is raised or mound shaped. The raised or mound shaped portion around one or more of the holes 326 and/or the offset holes 336 can increase a strength of the bone surgical plate device 300 at or about the one or more of the holes 326 and/or the offset holes 336 decreasing failure of the bone surgical plate device 300 at or about the one or more of the holes 326 and/or the offset holes 336.

In some cases, the bone surgical plate devices 300 and 300A can include stainless steel, cobalt alloy, bioceramic, titanium, titanium alloy, carbon fiber reinforced polymers, and/or any other material commonly used for bone surgical plates.

In some cases, edges of the bone surgical plate devices 300 and 300A can be rounded or beveled to provide more patient comfort.

In some cases, a bottom surface of the bone surgical plate devices 300 and 300A can be contoured to include a same or similar shape to a surface of the clavicle bone and/or any other bone, wherein the bottom surface is a surface of the bone surgical plate devices 300 and 300A that contact the surface of the clavicle bone and/or any other bone. In some cases, at least a portion of the bottom surface can not contact the surface of the clavicle bone and/or any other bone to prevent restriction of blood flow to the bone.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and configurations of the embodiments may be made and still fall within the scope of the disclosure. For example, features described above in connection with one embodiment can be used with a different embodiment described herein and the combination still fall within the scope of the disclosure. It should be understood that various features and configurations of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some cases, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain configurations, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 2 degrees, 1 degree, 0.1 degree, or otherwise. Additionally, as used herein, “gradually” has its ordinary meaning (e.g., differs from a non-continuous, such as a step-like, change).

It will be appreciated that the terms anterior, posterior, superior, inferior, medial, lateral, top, bottom, front, back, left and right are meant to provide context for the orientation and perspective for the embodiments described herein but should not be limiting on the intended placement of the described devices. Therefore, in some embodiments, anterior and posterior may be reversed, superior and inferior may be reversed, medial and lateral may be reversed, top and bottom may be reversed, front and back may be reversed, and/or left and right may be reversed, depending on the anatomy.

The scope of the present disclosure is not intended to be limited by the specific disclosures of preferred embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A clavicle plate fixation system, the system comprising:
a plate sized for positioning on a clavicle bone, the plate comprising:
a central fastening segment;
a medial connection segment coupled to a medial end of the central segment, the medial connection segment comprising a hole comprising two connected holes;
a lateral connection segment coupled to a lateral end of the central fastening segment, the lateral connection segment comprising a hole comprising two connected holes;
a medial fastening segment coupled to a medial end of the medial connection segment, the medial fastening segment comprising a first hole at a medial end of the medial fastening segment, a second hole at a lateral end of the medial fastening segment, and a third hole wherein the third hole is offset from the first hole and the second hole such that a portion of the third hole is past a first side of the medial fastening segment;
a lateral fastening segment coupled to a lateral end of the lateral connection segment the lateral fastening segment comprising a fourth hole at a lateral end of the lateral fastening segment, a fifth hole at a medial end of the lateral fastening segment, and a sixth hole wherein the sixth hole is offset from the fourth hole and the fifth hole such that a portion of the sixth hole is past a second side of the medial fastening segment;
wherein the medial fastening segment and the medial connection segment are separated by an indentation on a first side of the plate and an indentation on a second side of the plate, and the lateral fastening segment and the lateral connection segment are separated by a second indentation on the first side of the plate and a second indentation on the second side of the plate, and
wherein the medial connection segment and the central fastening segment are separated by a third indentation on the first side of the plate and a third indentation on the second side of the plate, and the lateral connection segment and the central fastening segment are separated by a fourth indentation on the first side of the plate and a fourth indentation on the second side of the plate,
wherein the third hole is offset from the first hole about a first axis by an angle of 45 degrees to 135 degrees, wherein the first axis extends from the lateral end of the medial fastening segment to the medial end of the medial fastening segment, and the sixth hole is offset from the fourth hole about a second axis by an angle of −45 degrees to −135 degrees, wherein the second axis extends from the medial end of the lateral fastening segment to the lateral end of the lateral fastening segment and wherein the plate is s-shaped along a longitudinal axis, wherein the longitudinal axis runs through the central fastening segment along a length of the plate.

2. The clavicle fixation plate system of claim 1, wherein the plate is configured to be bent at a location of the indentations.

3. The clavicle fixation plate system of claim 1, wherein the plate is configured to be coupled to a clavicle on a superior surface of the clavicle.

4. The clavicle fixation plate system of claim 1, wherein the central fastening segment comprises one or more screw holes.

5. The clavicle fixation plate system of claim 1, wherein each of the holes is a screw hole.

6. The clavicle fixation plate system of claim 1, wherein the plate comprises a thickness between 2 mm and 4 mm.

7. A fixation plate system comprising:
a plate sized for positioning on a bone, the plate comprising:
at least three fastening segments separated by an indentation on a first side of the plate and an indentation on a second side of the plate, each of the three fastening segments comprising one or more holes,
wherein a medial segment of the at least three fastening segments comprises a first tab offset from a top surface of the medial segment by an angle of 10 degrees to 135 degrees, and a lateral segment of the at least three fastening segments comprises a second tab offset from a top surface of the medial segment by an angle of −10 degrees to −135 degrees.

8. The fixation plate system of claim 7, wherein the first tab extends from a first side of the plate in a first direction, and the second tab extends from a second side of the plate in a second direction generally opposite the first direction.

9. The fixation plate system of claim 8, wherein the medial segment does not comprise any tabs extending in the second direction from the second side of the plate.

10. The fixation plate system of claim 8, wherein the lateral segment does not comprise any tabs extending in the first direction from the first side of the plate.
